(19) United States
(12) Patent Application Publication
(10) Pub. No.: US 2012/0181189 A1
(43) Pub. Date: Jul. 19, 2012

(54) Amperometric Creatinine Biosensor With Immobilized Enzyme-Polymer Composition And Systems Using Same, And Methods

(75) Inventor: Stephen A. Merchant, Norman, OK (US)

(73) Assignee: FRESENIUS MEDICAL CARE HOLDINGS, INC., Waltham, MA (US)

(21) Appl. No.: 13/497,083

(22) PCT Filed: Aug. 18, 2010
(86) PCT No.: PCT/US10/45824

§ 371 Date: Mar. 20, 2012

Related U.S. Application Data
(60) Provisional application No. 61/245,403, filed on Sep. 24, 2009.

(51) Int. Cl. G01N 27/327; B05D 5/00; B05D 3/00; G01N 33/70

(52) U.S. Cl. 205/777.5; 204/403.14; 204/403.1; 427/58

(57) ABSTRACT

An amperometric biosensor is provided for determination of creatinine in a sample fluid. The biosensor can be an enzyme-polymer composition having at least one redox polymer and a plurality of enzymes immobilized on an electrode surface. Methods of preparing the amperometric biosensor are included. In addition, methods and systems using the amperometric biosensor in measuring creatinine concentrations of a patient and treatments of a patient with monitoring of the progress of dialysis performed on the patient are also provided.

This application claims the benefit under 35 U.S.C. §119(e) of prior U.S. Provisional Patent Application No. 61/245,403, filed Sep. 24, 2009, which is incorporated in its entirety by reference herein.

### BACKGROUND OF THE INVENTION

The present invention relates to an amperometric biosensor for determining the concentration of creatinine or other analytes in a sample fluid. In particular, the present invention relates to amperometric biosensors having an immobilized enzyme-redox polymer composition as a creatinine concentration sensing element attached to an electrode, and methods of preparing these biosensors. The present invention also relates to methods and systems for using these amperometric biosensors to measure creatinine or other analyte concentrations in a sample fluid.

A biosensor is an analytical device incorporating biological and chemical sensing elements, either intimately connected to or integrated with a suitable transducer, which enables the conversion of concentrations of specific chemicals into electronic signals. Biosensors have been produced that incorporate an enzyme as a biological recognition component. Mediators are also frequently used in the biosensor as electron shuttles or carriers to enhance electron transfer between the enzymes and electrode surface. As such, the mediators must be able to contact both the enzyme and electrode surface; and hence, can be added to biosensors by physically mixing the mediator with the enzyme, by chemically binding the mediator to the enzyme, or by separating the mediator and enzyme by a semi-permeable membrane such that the two moieties are in contact and both in the liquid phase, but not physically mixed together.

Biosensors have been used to detect biological analytes such as creatinine. Creatinine is the naturally produced final product of creatine metabolism in mammals. Creatinine is filtered from the bloodstream by the kidneys in relatively constant amounts every day. During kidney dysfunction or muscle disorder, the creatine concentration in serum/plasma may rise to levels many times the norm. The measurement of the creatinine levels in serum and the determination of the renal clearance are used for laboratory diagnosis of renal and muscular function. Some previous creatine measurements have been based on colorimetry. These methods are analytically limited as not being specific for creatinine. Given this, many other substrates interfere with the assay leading to inaccurate determinations of creatine concentration in the sample. Potentiometric biosensors for creatinine measurements also are known, which are unstable due to potential drift. Amperometric biosensors for creatinine also have been developed that require use of a diffusional mediator, such as oxygen, hydrogen peroxide, or various synthetic mediators. As presently understood, amperometric biosensors using diffusional mediators tend to produce a small current, high interference signal, which makes transduction, measurability, and precision more difficult. Also, as presently understood, changes in dissolved oxygen, temperature, pH, or other stimuli can have a destabilizing effect on the selectively and accuracy of a biosensor using a diffusional mediator.

### SUMMARY OF THE INVENTION

The present invention relates to an amperometric biosensor for determination of creatinine or other analytes in a sample fluid. The biosensor comprises an immobilized enzyme-polymer composition and an electrode having a surface to which the enzyme-polymer composition is attached. The enzyme-polymer composition comprises a redox polymer and a plurality of enzymes crosslinked on the electrode surface. The enzymes include a redox enzyme and at least one enzyme catalyzing hydrolysis of creatinine or a hydrolyzed derivative thereof. The redox polymer provides direct electrical communication between the redox enzyme and the electrode surface.

It has been found that an amperometric biosensor for creatinine can be provided with a non-diffusing redox polymer as an electron mediator integrated with enzymes (e.g., redox and hydrolase enzymes) and an electrode, to ensure direct electron transfer between a redox enzyme and the electrode. The hydrolase enzyme(s) converts creatinine to an oxidizable substrate that is a source of electrons that are transferred to the redox polymer by a redox enzyme, and, from there, are directly transferred to the electrode, which is held in a narrow range of potentials. This scheme of enzymes and redox polymer makes the biosensor selective to only the particular analyte of interest, creatinine. The redox polymer permits the direct measurement of the current generated by the enzymatic reaction, without reliance on the diffusion of mediators from the bulk solution or surroundings. Changes in dissolved oxygen, temperature, pH, or other stimuli, that can have a destabilizing effect on the selectivity, responsiveness, and accuracy of the biosensor, can be avoided in the present biosensors. Unlike diffusional mediators, the redox polymer concentration in the present composition remains constant. The mechanism by which current can be generated at the electrode in a biosensor of the present invention is by the oxidation of a creatinine derived substrate (e.g., sarcosine) generated by the cascade of enzymatic reactions. The resulting current correlates directly and is proportional to the creatinine concentration. Further, instead of an electrode needing to operate at a high potential range as in some biosensors using diffusional mediators (e.g., 600 mV), the electrode of the amperometric biosensor of the present invention can be operated at significantly reduced potential range (e.g., from about 350 mV to about 400 mV). Also, a higher current response over a wider range of substrate concentrations can be provided with the present biosensor. The biosensors of the present invention can be miniaturized for ease of use, including, for example, for analyzing a medical treatment fluid stream, such as a medical treatment fluid that has been interacted with a biological fluid or biological component(s) thereof, such as a dialysate stream, or at another point of care for directly analyzing a sample of biological fluid (e.g., using a handheld meter equipped with the biosensor).

The present invention also relates to a method for preparing an amperometric biosensor for determination of creatinine in a sample fluid comprising depositing an aqueous mixture containing a plurality of enzymes including a redox enzyme and at least one enzyme catalyzing hydrolysis of creatinine or a hydrolyzed derivative thereof, a redox polymer, and a crosslinker on a surface of an electrode, and crosslinking the aqueous mixture to form an enzyme-polymer composition immobilized on the electrode surface.

The present invention additionally relates to a method of detecting or monitoring creatinine concentration in a sample fluid, comprising contacting a present biosensor with a sample fluid, measuring current at the electrode, and correlating the measured current with creatinine concentration in the sample fluid. The redox polymer directly transfers electrons generated by the enzymatic reaction to the electrode of the biosensor. The current resulting from the electron transfer is directly proportional to the creatinine concentration in the sample fluid. The sample fluid can be a medical treatment fluid, such as a medical treatment fluid that has been interacted with a biological fluid or biological component(s) thereof, such as, for example, a dialysate stream. The sample fluid also can be a biological fluid such as, for example, blood (whole blood, plasma, or serum), urine, or saliva.

The present invention further relates to a method of treating an animal, comprising contacting a present biosensor with a fluid stream of a dialyzer used in the dialysis of the animal for measuring the creatinine concentration in the fluid stream in real time. The fluid stream of the dialyzer can be, for example, a dialysate stream, an arterial line, a venous line, or any combination thereof. The biosensor can be deployed, for example, in a dialysate stream, post-dialyzer or pre-cartridge. The biosensor can be used to determine a patient's initial creatinine concentration, the progress of creatinine clearance during dialysis treatment, or both, in real time. The dialysis of the blood of the animal can be discontinued after the measured creatinine concentration reaches a pre-selected target value. The creatinine concentration measurement can be performed in the dialysate stream in real time continuously, semi-continuously, or intermittently. Further, where a correlation exists between creatinine and another chemical of interest, then it may be possible to determine these levels and clearance indirectly yet accurately with the biosensor. For example, the concentration of creatinine measured may be correlated to BUN (blood urea nitrogen), for evaluation of kidney function and/or monitoring the effectiveness of dialysis.

It is to be understood that both the foregoing general description and the following detailed description are exemplary and explanatory only and are only intended to provide a further explanation of the present invention, as claimed.

The accompanying drawings, which are incorporated in and constitute a part of this application, illustrate some of the embodiments of the present invention and together with the description, serve to explain the principles of the present invention. The drawings are not necessarily drawn to scale.

### BRIEF DESCRIPTION OF THE DRAWINGS

FIG. 1 is a reaction schematic for determination of a creatinine level with a biosensor using a diffusional electron mediator.

FIG. 2 is a reaction schematic for determination of a creatinine level with an amperometric biosensor using a non-diffusional redox polymer as the electron mediator.

FIG. 3 shows a multi-staged electrochemical reaction pathway including stages (a), (b), (c), (d), and (e), for enzymatic determination of creatinine.

FIG. 4 is a schematic of an amperometric creatinine biosensor electrode having multiple enzymes immobilized in a redox polymer that is crosslinked as a coating attached to an electrode.

FIG. 5 is an amperometric biosensor for creatinine in a test strip configuration in plan view.

FIG. 6 is an amperometric biosensor for creatinine in cross-sectional view along line 5′-5′ of FIG. 5.

FIG. 7 is a dialysis system including an amperometric biosensor for creatinine.

### DETAILED DESCRIPTION OF THE PRESENT INVENTION

The present invention relates to an amperometric creatinine biosensor that uses a non-diffusing redox polymer to mediate electron exchange between a biological element comprising enzymatic biocatalysts, and an electrode or transducer surface. A series of hydrolase and redox enzymes can be used to convert creatinine into a form from which an electrical current can be generated and carried by the redox polymer to an electrode where it is detected and converted into a measurable signal. The enzymatic biocatalysts are attached to the electrode surface with the crosslinked redox polymer. The redox polymer functions as a non-diffusing electron mediator and as a crosslinkable carrier material for immobilizing active enzymes and the redox polymer at the electrode surface.

Methods also are provided for detecting or monitoring creatinine concentration in a sample fluid. The sample fluid can be, for example, a treatment fluid associated with the administration of a medical or therapeutic treatment to a patient (e.g., a dialysate stream fluid), or a patient's own biological fluid. The fluid usually is a liquid unless indicated otherwise, although gases are also contemplated and not excluded. A sample fluid is contacted with a biosensor comprising an enzyme-polymer composition comprising a redox polymer and a plurality of enzymes immobilized on an electrode surface. The redox polymer transfers electrons generated by enzymatic reactions supported by the enzymes to the electrode of the biosensor. The current resulting from the electron transfer is directly proportional to the creatinine concentration in the sample fluid. The ability to operate the amperometric biosensor with a non-diffusional redox polymer mediator at optionally reduced potentials (e.g., from about 350 to about 450 mV, or other values), permits higher current output, better signal acquisition and less noise (i.e., increased signal/noise (SN)), a wider range of detection (substrate concentrations), and/or enhanced selectivity to creatinine present in the sample fluid. As stated above, the sample fluid can be medical treatment fluid, such as, for example, a dialysate stream fluid (e.g., post-dialyzer sample or pre-cartridge sample). If the sample fluid for analysis with the present biosensor is a biological fluid, it can be, for example, blood (whole blood, plasma, or serum), urine, or saliva. The sample fluid can be monitored for creatinine concentration during the course of a medical treatment, for example, during dialysis, or at a point of care by testing a biological fluid (e.g., using a glucose meter on a body fluid).

The present invention includes the following aspects/embodiments/features in any order and/or in any combination:

1. The present invention relates to an amperometric biosensor for determination of creatinine in a sample fluid comprising an enzyme-polymer composition and an electrode having a surface, wherein the enzyme-polymer composition comprises at least one redox polymer and a plurality of enzymes immobilized on the electrode surface, and wherein the enzymes comprise at least one redox enzyme and at least one enzyme catalyzing hydrolysis of creatinine or a hydrolyzed derivative thereof.

2. The biosensor of any preceding or following embodiment/feature/aspect, wherein the enzyme-polymer composition is a coating on the surface of the electrode.

3. The biosensor of any preceding or following embodiment/feature/aspect, where the redox polymer is attached to the enzymes and the electrode surface of the biosensor through crosslinking.

4. The biosensor of any preceding or following embodiment/feature/aspect, wherein the redox polymer comprises a neutral polymeric backbone and redox active moieties attached thereto.

5. The biosensor of any preceding or following embodiment/feature/aspect, wherein the redox polymer comprises a neutral polymeric backbone and redox active moieties attached thereto, wherein the redox moieties comprise organometallic species comprising a transition metal.

6. The biosensor of any preceding or following embodiment/feature/aspect, wherein said redox polymer is X-poly(vinylpyridine), X-poly(vinylimidazole), X-poly(allylamine), or X-poly(ethyleninime) or any combination thereof, where X is at least one organometallic moiety comprising a transition metal that is iron, osmium, ruthenium, or cobalt or any combination thereof.

7. The biosensor of any preceding or following embodiment/feature/aspect, wherein said plurality of immobilized enzymes comprise creatinine amidohydrolase, creatine amidinohydrolase, and sarcosine oxidase.

8. The biosensor of any preceding or following embodiment/feature/aspect, wherein said composition contains from about 1 wt % to about 99 wt % redox polymer, from about 1 wt % to about 99 wt % enzymes, and from about 1 wt % to about 30 wt % crosslinker, wherein the enzymes comprise creatinine amidohydrolase, creatine amidinohydrolase, and sarcosine oxidase.

9. The biosensor of any preceding or following embodiment/feature/aspect, wherein said biosensor comprises at least one working electrode, at least one reference electrode and at least one counter electrode.

10. The biosensor of any preceding or following embodiment/feature/aspect, wherein said biosensor comprises at least one working electrode, at least one reference electrode and at least one counter electrode, and wherein the enzyme-polymer composition is applied to said working electrode.

11. A dialysis system comprising the amperometric biosensor of any preceding or following embodiment/feature/aspect.

12. An immobilized enzyme-polymer composition for an electrode surface comprising at least one crosslinked redox polymer and a plurality of enzymes comprising at least one redox enzyme and at least one enzyme catalyzing hydrolysis of creatinine or a hydrolyzed derivative thereof.

13. A method for making the amperometric biosensor of any preceding or following embodiment/feature/aspect comprising:

depositing an aqueous mixture containing said plurality of enzymes, at least one redox polymer and at least one crosslinker on a surface of said electrode; and

crosslinking the mixture to form said enzyme-polymer composition immobilized on the electrode surface.

14. The method of any preceding or following embodiment/feature/aspect, wherein said plurality of enzymes comprise creatinine amidohydrolase, creatine amidinohydrolase, and sarcosine oxidase.

15. The method of any preceding or following embodiment/feature/aspect, wherein said redox polymer is X-poly(vinylpyridine), X-poly(vinylimidazole), X-poly(allylamine), or X-poly(ethyleninime) or any combination thereof, where X is at least one organometallic moiety comprising a transition metal that is iron, osmium, ruthenium, or cobalt, or any combination thereof.

16. A method of detecting creatinine concentration in a sample fluid, comprising:

contacting the biosensor of claim 1 with a sample fluid;

measuring current at the electrode; and

correlating the measured current with creatinine concentration in the sample fluid.

17. The method of any preceding or following embodiment/feature/aspect, wherein the sample fluid is a dialysate stream.

18. The method of any preceding or following embodiment/feature/aspect, wherein the sample fluid is a biological fluid.

19. The method of any preceding or following embodiment/feature/aspect, wherein the electrode is operated at from about 350 to about 400 mV.

20. A method of treating an animal for clearance of creatinine, comprising:

contacting the biosensor of claim 1 with a fluid stream of a dialyzer used in the dialysis of an animal; and

measuring creatinine concentration in said fluid stream of the dialyzer with the biosensor.

21. The method of any preceding or following embodiment/feature/aspect, wherein the fluid stream used for the creatinine concentration measurement is a dialysate stream.

22. The method of any preceding or following embodiment/feature/aspect, wherein the fluid stream used for the creatinine concentration measurement is a post-dialyzer dialysate stream.

23. The method of any preceding or following embodiment/feature/aspect, where the creatinine concentration measurement is done in real time continuously, semi-continuously, or intermittently.

24. The method of any preceding or following embodiment/feature/aspect, further comprising discontinuing dialysis treatment on the animal after a measured creatinine concentration reaches a pre-selected target value.

The present invention can include any combination of these various features or embodiments above and/or below as set forth in sentences and/or paragraphs. Any combination of disclosed features herein is considered part of the present invention and no limitation is intended with respect to combinable features.

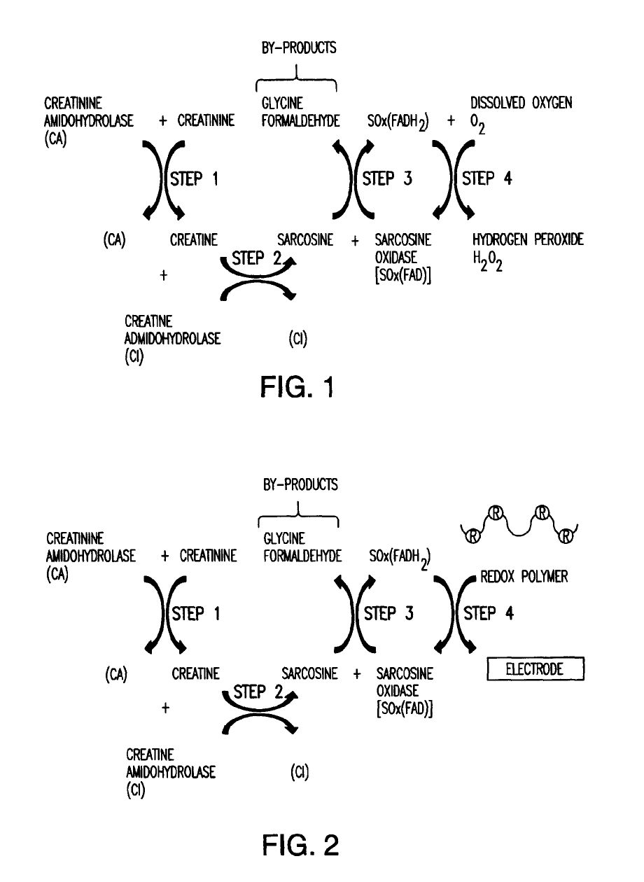

Amperometric biosensor schemes. FIG. 1 shows a reaction scheme of a creatinine biosensor using a diffusional mediator for sake of comparison. FIG. 2 shows one example of a reaction scheme of an amperometric creatinine biosensor using a non-diffusing redox polymer mediator as part of an enzyme-polymer composition immobilized on an electrode according to the present invention.

As shown in FIG. 1, the presence of creatinine is detected by biosensor measurement of concentration changes of hydrogen peroxide formed in the final reaction. Creatinine is hydrolyzed to creatine, which itself is hydrolyzed to sarcosine. Hydrolase enzymes are used for this series of transformations. Sarcosine is then oxidized using a redox enzyme, sarcosine oxidase, to form the by-products of glycine and formaldehyde. When sarcosine is oxidized, a FAD site on the sarcosine oxidase enzyme is reduced. The enzymes, creatinine amidohydrolase or creatininase (CA) and creatine amidinohydrolase or creatinase (CI), remain unchanged by the reactions, whereas the flavin-containing enzyme sarcosine oxidase SOx(FAD) is reduced to SOx(FADH₂). FAD and FADH₂ are the oxidized and reduced forms of flavin adenine dinucleotide (FAD), respectively. The sarcosine oxidase enzyme is then regenerated by reducing dissolved oxygen to hydrogen peroxide. Oxygen is the electron mediator in this biosensor scheme. The sarcosine oxidase takes electrons from the sarcosine (oxidizes the sarcosine) and then oxygen essentially takes those electrons from the enzyme and carries them to the electrode in the form of hydrogen peroxide. This biosensor scheme of FIG. 1, however, is inefficient because of diffusional limitations and a requirement for precise control of oxygen concentration in the system, particularly if it is in vitro. It is inconvenient, for example, to ensure that oxygen concentration in a series of blood samples is maintained at a constant. Additionally, the electrodes for hydrogen peroxide detection require high operating potentials (e.g., approximately 600 mV), which may cause blood metabolites, such as ascorbic acid or uric acid, or acetaminophen, or other species, to be oxidized at the electrodes, thus leading to inaccurate measurements as the selectivity of the sensor to only hydrogen peroxide is reduced. This type of sensor scheme is inefficient and suffers from several potential diffusional limitations. The diffusional mediator oxygen must diffuse to the redox enzyme and the hydrogen peroxide must diffuse to the electrode surface. The result is a sensor with a low current output, high noise, and reduced signal.

FIG. 2 shows the presence of creatinine as detected by an amperometric biosensor measurement where the concentration of creatinine is measured using at least one non-diffusing redox polymer as an electron mediator attached to hydrolase and redox enzymes and an electrode surface through crosslinking. This arrangement provides direct electrical communication between the redox enzyme and the electrode. Creatinine is initially hydrolyzed to creatine, similar to the reaction scheme of FIG. 1, which itself is hydrolyzed to sarcosine. Hydrolase enzymes are used for this series of transformations. Sarcosine is then oxidized to form the by-products of glycine and formaldehyde. The redox enzyme, SOx(FAD), oxidizes the substrate (sarcosine). When the sarcosine is oxidized, the FAD site on the sarcosine oxidase enzyme is reduced. The enzyme creatinine amidohydrolase (CA) and creatine amidinohydrolase (CI) remain unchanged by the reactions. The flavin-containing enzyme sarcosine oxidase SOx(FAD) is reduced to SOx(FADH₂) as the sarcosine is converted into the glycine and formaldehyde by-products. Unlike the reaction scheme of FIG. 1, sarcosine oxidase enzyme is then regenerated by interaction with a redox polymer present in the enzyme-polymer composition immobilized on the electrode. For example, the redox enzyme, SOx(FADH₂), donates the electrons to the redox active species on the polymer. The redox polymer then donates the electrons to the electrode. That is, the redox polymer takes the electrons from the oxidized redox enzyme and directly shuttles them to the electrode. The redox polymer is the electron mediator in this biosensor scheme of FIG. 2.

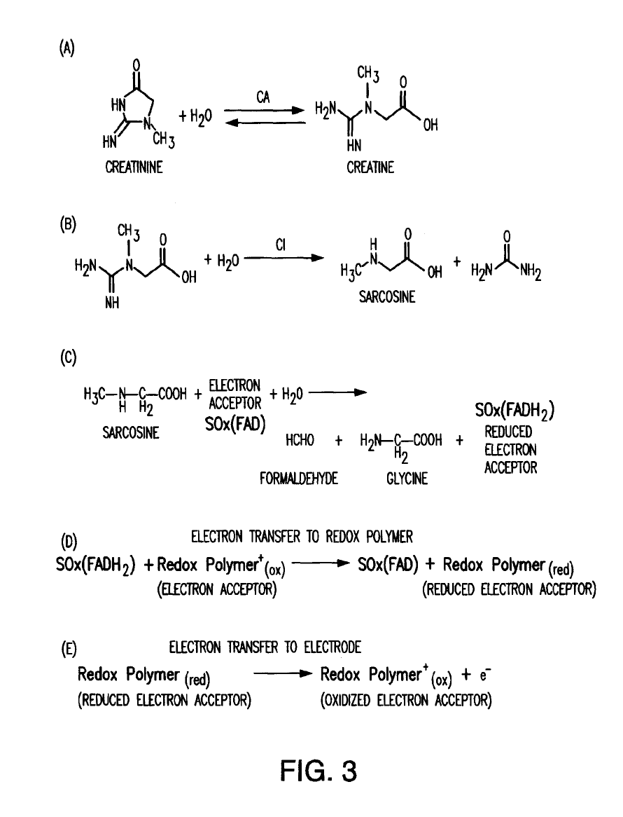

Reaction pathways of amperometric biosensing. FIG. 3 shows a multi-staged electrochemical reaction pathway including stages (a), (b), (c), (d), and (e), for enzymatic determination of creatinine as shown in the reaction scheme of FIG. 2. Reactions (a), (b) and (c) are enzyme-catalyzed reactions. Stage (d) represents the transfer of electrons from the redox enzyme to one or more redox moieties on the redox polymer. Stage (e) represents the direct transfer of the electrons from one or more redox moieties on the redox polymer to the electrode.

This amperometric biosensor scheme and associated reaction pathways, such as illustrated in FIGS. 2-3, are more efficient than biosensor systems using a diffusional electron mediator, such as illustrated in FIG. 1. The polymer location is fixed relative to the enzymes and the electrode surface, and its concentration is held essentially constant, to avoid destability that can be associated with use of a diffusional mediator. The system such as in FIG. 2 also avoids or reduces possible affects of uninvited mediator sources that can reduce the sensor selectivity and measurement accuracy. The electrodes for this system, such as illustrated in FIG. 2, can operate at reduced potentials, such as from about 100 mV to about 550 mV, or from about 200 mV to about 500 mV, or from about 300 mV to about 450 mV, or from about 350 mV to about 400 mV.

Enzyme-polymer composition. The present enzyme-polymer composition contains enzymes and at least one redox polymer. The enzymes include at least one redox enzyme and at least one enzyme catalyzing hydrolysis of creatinine or a hydrolyzed derivative thereof. As stated above, the enzymes support the biocatalytic conversion of creatinine to a form from which electrons can be transferred from a redox enzyme to the redox polymer. From there, the electrons are shuttled directly from the redox polymer to an electrode where they are detected as an electrical current. The redox polymer is the component of an enzyme-polymer composition that attaches the enzymes to an electrode surface of the biosensor. In an example, a redox enzyme, sarcosine oxidase, and hydrolase enzymes (e.g., amidohydrolase and amidinohydrolase), are immobilized on the working electrode with the redox polymer crosslinked thereupon. The redox polymer attaches to the enzymes by chemical bonding, such as by crosslinking, hydrogen bonding, covalent binding, matrix inclusion, or any a combination thereof; or by a physical interaction, such as electrostatic or hydrophobic interactions. All of the biosensing composition components (redox polymer and enzymes) are immobilized on the electrode. The immobilized enzyme-polymer composition can be, for example, in the form of a dry, solid, enzyme crosslinked product that is a stable composition.

Redox Polymer. The redox polymer of the electrocatalyst coating, film or layer electrically connects, or “wires,” the reaction centers of the redox enzyme to the electrode surface. This electrical connection or “wiring” can be accomplished without using a diffusing mediator. In one example, the redox polymer is a crosslinkable polymer, which when crosslinked onto the electrode surface immobilizes the biosensing components thereon. One or more redox polymers can be used.

In an example, the redox polymer is a polycation that forms an electrostatic adduct with the redox enzyme. In one particular example, the redox polymer is used in the electrical connection of sarcosine oxidase to the electrode surface. In one example, a suitable redox polymer is one having a redox species that is a transition metal compound or complex. A particular example of a transition metal compound or complex is one in which the transition metal is osmium, ruthenium, iron, or cobalt. The redox polymer can comprise a neutral polymeric backbone and redox active moieties attached thereto. The present redox polymer is different than an electroconductive polymer that carries a charge along its backbone. The redox active moieties can undergo oxidation (donate electrons) and reduction (accept electrons). The redox polymer can be selected, for example, from X-poly(vinylpyridine), X-poly(vinylimidazole), X-poly(allylamine), and/or X-poly(ethyleninime), where X is an organometallic moiety comprising a metal, such as one selected from iron, osmium, ruthenium, or cobalt. The organometallic moiety can be, for example, a pendant or branched unit that repeats at regular or irregular intervals along the backbone of the polymer. Sufficient organometallic moieties can be provided in the polymer to support electron shuttling between SOx(FADH₂) and the electrode surface at a level where a current signal can be acquired that can be correlated with creatinine concentration in the analyte.

The redox polymer can have several desirable characteristics, including but not limited to, a flexible, hydrophilic neutral (charge) backbone, which provides segmental mobility when the redox polymer is hydrated, and redox functions pendant on flexible and hydrophilic spacers, which tend to maximize electron exchange between the colliding redox centers. Further, small complexes of transition metals, such as Os^{2+/3+}, having high rates of self-exchange, which small complexes allow the close approach of the reaction centers of the enzymes. For example, an exemplary redox polymer is PVP-[Os(N,N′-dialkylated-2,2′-bi-imidazole)₃Cl]^{2+/3+}. Another exemplary redox polymer is osmium-poly(4-vinylpyridine), which can be synthesized by partially complexing the pyridine nitrogens of poly(4-vinylpyridine) with Os(bpy)₂Cl^{+/2+} and then partially quaternizing the resulting polymer with 2-bromoethylamine according to a previously published protocol (Gregg, B. A. et al., Anal. Chem., 1990, 62 (3), 258-263). Additional redox polymers include, for example, poly(l-vinyl imidazole); poly(4-vinyl pyridine); or copolymers of 1-vinyl imidazole such as poly (acrylamide co-1-vinyl imidazole) where the imidazole or pyridine complexes with [Os (bpy)₂Cl]^{+/2+}; [Os (4,4′-dimethyl bipyridine)₂Cl]^{+/2+}; [Os (4,4′-dimethyl phenanthroline)₂Cl]^{+/2+}; [Os (4,4′-dimethyoxy phenanthroline)₂Cl]^{+/2+}; and [Os (4,4′-dimethoxy bipyridine)₂Cl]^{+/2+}; to imidazole rings. These forms of redox polymers are illustrated, for example, in U.S. Pat. Nos. 5,543,326, 5,593,852, and U.S. Pat. Appln. Publ. No. 2008/0118782 A1, all of which are incorporated in their entireties herein by reference. Another exemplary redox polymer is ferrocene-modified poly(ethylenimine), such as ferrocene-carboxaldehyde linear or branched poly(ethylenimine), which can be synthesized, for example, according to previously published protocol (Merchant, S., et al., Langmuir 2007, 23, 11295-11302). Although having been illustrated here as a redox polymer having a redox species that is a transition metal compound or complex, in other examples, the redox polymer can be, for example, an electronically conductive polymer (ECP), such as, for example, polypyrrole.

The redox polymer, such as a redox polymer having a redox species that is a transition metal compound or complex, is not limited to any molecular weight (M_{w}) or number average molecular weight (Mₙ). The polymer can have, for example, an average M_{w} in a range of from about 1 kilodalton (kDa) to about 1000 kDa or more, and a Mₙ can be in a range of from about 1 kDa to about 100 kDa or more. Other molecular weights and number average molecular weights are possible.

Sufficient redox polymer is provided in the enzyme-polymer composition to immobilize the components of the composition when crosslinked to an electrode surface, and to support the electron shuttling between the redox polymer and electrode surface. In one example, the amount of redox polymer in the enzyme-polymer composition is from about 10 wt % to 80 wt %, or from about 25 wt % to about 65 wt %, or from about 40 wt % to about 50 wt %, based on the weight of the composition.

Enzymes. The enzymes comprise hydrolase enzymes and a redox enzyme adequate to support the cascade of reactions such as shown in FIG. 2. In the provided illustration, the hydrolase enzymes include creatinine amidohydrolase and creatine amidinohydrolase, and the redox enzyme is sarcosine oxidase (SOx). Other enzymes having similar biocatalytic interactions or affects on creatinine or its hydrolyzed derivatives also can be used. The enzyme creatinine amidohydrolase and creatine amidinohydrolase remain unchanged by the reactions. Sarcosine oxidase (SOx) catalyzes the oxidative demethylation of sarcosine (N-methylglycine) and forms formaldehyde and glycine. In one example, the redox enzyme of sarcosine oxidase has a prosthetic group, which can exchange electrons, such as, for example, FAD, NAD (nicotinamide adenine dinucleotide), quinone, and the like. A flavin-containing enzyme SOx(FAD) from the Arthrobacter sp. is a monomer. The monomeric sarcosine oxidases are flavine proteins that contain a mole of flavine adenine dinucleotide (FAD) that is covalently linked to the enzyme by a cysteine residue. As illustrated in FIGS. 2-3, the flavin-containing enzyme sarcosine oxidase SOx(FAD) is reduced to SOx(FADH₂) when the sarcosine is converted into the glycine and formaldehyde by-products.

Creatinine amidohydrolase, creatine amidinohydrolase, and sarcosine oxidase can be commercially obtained. Creatinine amidohydrolase is available, for example, as creatininase from Pseudomonas sp. (C3172)(100-300 units/mg protein, where one unit can hydrolyze 1.0 mmole CA to CI per min. at pH 8.0 and 25° C.), or creatininase from Flavobacterium sp. (C7399)(150-400 units/mg protein, where one unit can hydrolyze 1.0 μmole CA to CI per min. at pH 6.5 and 37° C.), from Sigma-Aldrich. Creatine amidinohydrolase is available, for example, as creatinase from Actinobacillus sp. (C2409)(20-40 units/mg protein, where one unit can hydrolyze 1.0 μmole CA to urea and sarcosine per min. at pH 7.5 and 37° C.), creatinase from Pseudomonas sp. (C3921)(10-15 units/mg protein, where one unit can hydrolyze 1.0 μmole CA to urea and sarcosine per min. at pH 7.5 and 37° C.), or creatinase from Flavobacterium sp. (C7024)(10-20 units/mg protein, where one unit can hydrolyze 1.0 μmole CA to urea and sarcosine per min. at pH 7.5 and 37° C.), from Sigma-Aldrich. Sarcosine oxidase is available, for example, as sarcosine oxidase from Bacillus sp. (S7897)(25-50 units/mg protein, where one unit can form 1.0 μmole formaldehyde from sarcosine per min. at pH 8.3 and 37° C.) or sarcosine oxidase from Corynebacterium sp. (S7759)(5-10 units/mg protein, where one unit can form 1.0 μmole formaldehyde from sarcosine per min. at pH 8.3 and 37° C.), from Sigma-Aldrich.

The enzymes retain enzymatic activity after immobilization with the crosslinked redox polymer on an electrode. The immobilized enzymes can retain, for example, from about 90 percent to about 100 percent activity, or at least 50 percent, at least 40 percent, at least 25 percent, or at least 10 percent of the enzymatic activity (e.g., SU/g) compared to a non-immobilized enzyme.

In one example, the hydrolase and redox enzymes are used in approximately equal amounts. The use of a different amount of one of the enzymes relative to the others can be done, although the biocatalytic reaction rate may be limited by some differences in the proportions of the enzymes contained in the composition. Sufficient enzymes are included in the composition to support the biocatalytic reactions used to electrochemically convert creatinine analyte present in a sample to a measurable electrical current that is proportional to the concentration of creatinine. In one example, the amount of each individual hydrolase enzyme and the redox enzyme in the enzyme-polymer composition is present in an amount, for example, of from about 1 wt % to 99 wt %, or from about 2.5 wt % to about 75 wt %, or from about 5 wt % to about 50 wt %, or from about 10 wt % to about 30 wt %, based on the weight of the composition. The enzyme load proportion in the enzyme-polymer composition based on units (U) creatinine amidohydrolase (CA)/units (U) creatinine amidinohydrolase (CI)/units (U) sarcosine oxidase (SOx(FAD)), can be, for example, 17-27 U/11-21 U/0.5-1.5 U (CA/CI/SOx(FAD)), or 20-24 U/14-18 U/0.8-1.2 U (CA/CI/SOx(FAD)), or other ratios. The weight percentages of the enzymes CA, CI and SOx(FAD) enzymes provided in the enzyme-polymer composition can be, for example, adjusted to provide the above-illustrated unit proportions of the respective enzymes, or other proportions can be used.

In one example, it is contemplated that all three enzymes that are components of the illustrated system are immobilized in the enzyme-polymer composition. Only one or two of the three enzymes can optionally be immobilized into the enzyme-polymer composition. Thus, at least one or at least two or all three of the enzymes, creatinine amidohydrolase, creatine amidinohydrolase, and sarcosine oxidase, can be immobilized into the enzyme-polymer composition that is used in a present biosensor.

Crosslinker. At least one crosslinker can be included for crosslinking the redox polymer present in the enzyme-polymer composition. The crosslinked composition can have, for example, a three-dimensionally crosslinked structure. The crosslinker can comprise a bi- or poly-functional reagent which can form chemical bonds (e.g., covalent bonds) with the redox polymer. In one example, the crosslinker crosslinks the redox polymer at sites along polymer backbones other than the redox moieties, or at the redox moieties, or both. The crosslinker can form bonds with one or more of the enzymes. The crosslinker can comprise, for example, one or more epoxy groups, one or more acrylate groups, one or more halide groups, one or more carboxyl groups, one or more aldehyde groups, or any combinations thereof Suitable crosslinkers include, for example, a diacrylate- (or divinyl ether-) functionalized ethylene oxide oligomer or monomer. Examples of suitable crosslinkers are heterobifunctional polyethylene glycol, homobifunctional polyethylene glycol, or combinations thereof.

The crosslinker can have flexibility or segmental mobility. A crosslinker having flexibility can have a better opportunity to covalently bind to an enzyme and at the same time allows the enzyme to maintain and present an enzyme active site for the substrate. The crosslinker can be or comprise, for example, poly(ethylene glycol) diacrylate, tetra(ethylene glycol) diacrylate, poly(ethylene glycol) diglycidyl ether, tetra(ethylene glycol) divinyl ether, ethylene glycol diglycidyl ether, tetra(ethylene glycol) dimethacrylate, trimethylene glycol dimethacrylate, ethylene glycol dimethacrylate, dibromohexane, gluteraldehyde, epichlorohydrin, or any combination thereof.

The crosslinker can be present in the enzyme-polymer composition in an amount to crosslink at least a portion of the polymer and/or enzyme, such as from about 1 wt % to 25 wt %, or from about 1 wt % to 15 wt %, or from about 2 wt % to about 8 wt %, based on the weight of the composition. The crosslink density of the composition can be from 1% to 50% or more, such as from 9% to 50%, with respect to the % of the available functional groups on the polymer (e.g., nitrogen atoms on the polymer).

Exemplary enzyme-polymer formulation(s). In one example, the enzyme-redox polymer composition contains creatinine amidohydrolase, creatine amidinohydrolase, and sarcosine oxidase enzymes, such as in the above-illustrated unit proportions, and the redox polymer is a transition metal redox polymer, such as osmium poly(vinylpyridine). For this formulation, the composition contains from about 1 wt % to about 99 wt % redox polymer, from about 1 wt % to about 99 wt % total enzymes, and from about 1 wt % to about 30 wt % crosslinker, and, particularly, from about 20 wt % to about 80 wt % redox polymer, from about 20 wt % to about 80 wt % total enzymes, and from about 3 wt % to about 20 wt % crosslinker. The total enzymes comprised of creatinine amidohydrolase, creatine amidinohydrolase, and sarcosine oxidase enzymes also can be present in the stated enzyme-polymer composition, for example, in substantially equal gravimetric amounts (e.g., 0.9-1.1/0.9-1.1/0.9-1.1, w/w/w, (CA/CI/SOx(FAD)), or other proportions providing adequate active enzymatic activity to permit a biosensor current correlated to creatinine concentration in an analyte fluid to be detected and recorded.

The above-stated compositions can be used without need of any additional ingredients. The composition can further comprise additives, such as, for example, activated carbon, graphite, alumina, silica, or other high surface area, inert materials. The high surface area additives, such as activated carbon, or others, can function to increase active site surface area in the enzyme-polymer matrix, which can facilitate transfer of reaction current. As such high surface area materials also can tend to capacitively charge as an increasing function with their loading amount, which can cause signal noise. The amount of such high surface area materials, if used, accordingly should be controlled. An amount of activated carbon, graphite, alumina, silica, as used individually or in combinations, can be, for example, 0 to about 30 wt %, or from about 1 wt % to about 25 wt %, or from about 5 wt % to about 15 wt %, or other amounts. Additives, if used, preferably should not materially adversely affect the current output, SN, detection range of substrate concentrations, or the selectivity of the sensor for only creatinine in the sample analyte.

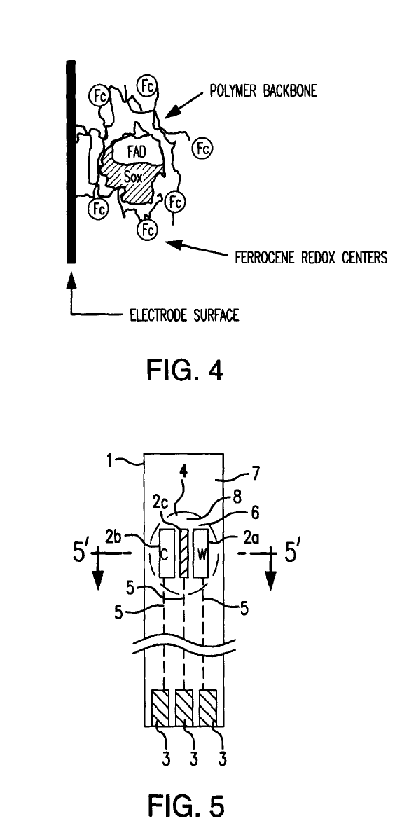

Method of preparation of biosensor electrode. Referring to FIG. 4, an amperometric biosensor fabricated by a method of an embodiment of the present invention is schematically shown. A ferrocene type redox center is indicated in FIG. 4 merely for illustration. Other transition metal type redox centers can be used in the redox polymers used in the present biosensors. Generally, an aqueous mixture containing the enzymes, the redox polymer, and crosslinking agent in an aqueous solution are applied on an electrode and dried or allowed to dry to form a sensing film or coating on the electrode surface. An aqueous mixture of the redox polymer, enzymes (hydrolase and redox enzymes), and crosslinker, is prepared. The order of addition and combination of these ingredients in an aqueous medium is not limited. One or more of the ingredients can be separately dispersed in separate aqueous solutions before their combination. In one example, an aqueous solution of each of the redox polymer and each enzymatic component, is prepared. The separate enzyme solutions and redox polymer are mixed together, such as to provide a uniform or substantially uniform mixture of the components, and a crosslinker is added to provide a biosensor precursor composition. In one example, each of the enzymes is uniformly or essentially uniformly distributed in the redox polymer in the enzyme-polymer precursor composition (composition before crosslinking), the enzyme-polymer composition after crosslinking, or both. When the enzymes and redox polymer are applied to the biosensor simultaneously from a single premixed source, it can be easier to control the uniformity of the composition. The enzymes and redox polymer also can be separately applied to the electrode surface from separate aqueous solutions. In one example, the enzyme-polymer composition is formed as a film, coating, sheet, or membrane on the surface of the electrode. The coating can be applied continuously or discontinuously over the exposed surface of an electrode. A sufficient coating can be applied to the electrode surface in a coverage and amount adequate for permitting detection of a current that can be correlated to a presence of creatinine in a sample contacted with the electrode. The concentrations of each separate solution can range, for example, from 0.1 mg/mL to 30 mg/mL, or from about 0.5 mg/mL to 20 mg/mL, or from about 1 mg/mL to 10 mg/mL, or other concentrations.

As stated above, an electrode surface or transducer is coated with the biosensor precursor composition and crosslinked to immobilize the composition on the electrode. The coating method is not limited, and can be, for example, dipping, immersion, solution casting, spin coating, spraying, and brushing. After application to the electrode, the coating or film is dried, such as by being allowed to dry at room temperature under a vacuum, such as for at least 1 hour, or at least 4 hours, or at least 8 hours, or at least 24 hours. The coated composition can be dried, for example, to a water content of from about 0 wt % to about 25 wt %, or from about 0 wt % to 15 wt %, 0.01 wt % to 5 wt %, or 0.1 wt % to 1 wt % based on the weight of the composition. A single coat or multiple coats can be applied to the electrode surface to build up to a total coating thickness. When using multiple coats, each coat can be the same or different with respect to thickness, amounts of each component, and the components themselves. The thickness of the film or coating formed on the electrode surface is not necessarily limited. The thickness of the film or coating can be, for example, from about 0.25 μm to about 500 μm, or from about 0.5 μm to about 250 μm, or from about 1 μm to about 100 μm, or from about 2 μm to about 50 μm.

The enzyme-polymer compositions and biosensor electrodes prepared according to the methods of the invention have considerable enzyme stability and allow for single or multiple-uses of the biosensor. The biosensor is enzymatically and operationally stable for at least one week, or at least 2 weeks, or at least 3 weeks, or at least 4 weeks, or at least 8 weeks.

Amperometric biosensor. A biosensor made with the enzyme-polymer composition of the present invention can be, for example, a multi-electrode configuration including a working electrode, a counter electrode, and a reference electrode. In one example, the enzymes are immobilized by crosslinking of the redox polymer on the working electrode. The working electrode can be, for example, carbon, glassy carbon, metal, metal oxides or a mixture of carbon and metal or metal oxides. In one example, the working electrode is a glassy carbon electrode. The reference electrode can be, for example, a saturated calomel reference electrode (SCE), Ag/AgCl, or saturated Hg₂Cl₂. The counter electrode can be, for example, a metal such as gold, silver, platinum or stainless steel, such as a metal wire counter electrode.

The biosensor electrodes, such as active electrodes, can be formed by coating a fine metal wire with the present enzyme-polymer formulation and drying the coating in place on the wire. For example, a fine platinum wire can be coated with the enzyme-formulation and the coating dried in place. The coated wire can be arranged in a syringe or other suitable flow cell or channel device that can be placed, for example, in-line or into the flow of an analyte stream to be monitored for creatinine concentration.

Platinum, silver, carbon, and Ag/AgCl ink also can be used in screen-printing methods, or photolithographically patterned metal vapor deposition methods, to form film sensors for the fabrication of miniaturized, planar, solid state electrodes. These electrodes can be used in electrode strips, biochips, and other miniaturized sensor configurations. The biosensor can be, for example, a screen-printed or photolithographically patterned three-electrode transducer with a platinum working electrode. Other transducer configurations also can be used.

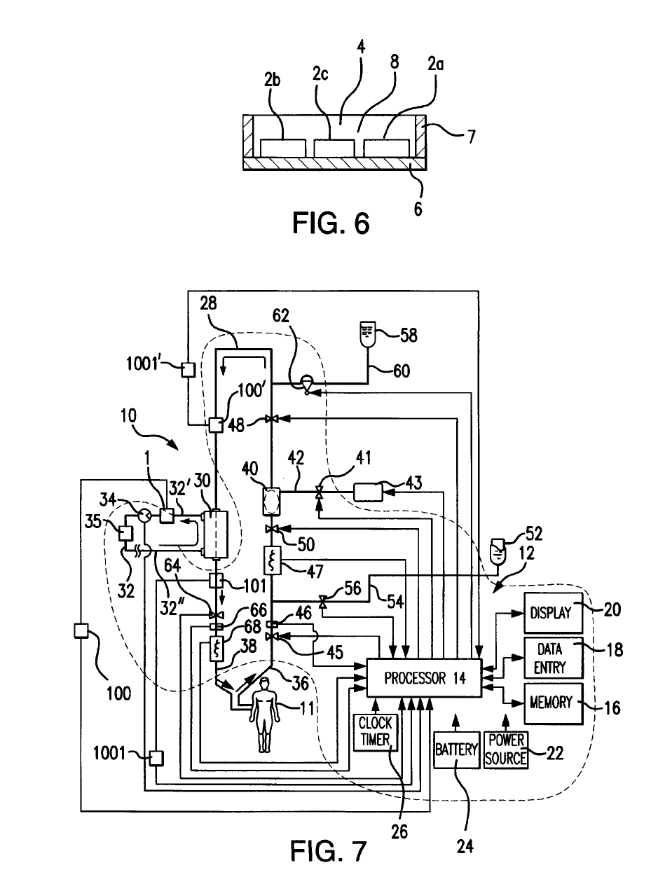

Referring to FIG. 5, in one example, an amperometric biosensor has the configuration of an electrode test strip 1 having an electrode support layer 6, an enzyme-redox polymer coated-working electrode 2a disposed on the support layer 6, and a counter electrode 2b and reference electrode 2c spaced from the working electrode 2a and disposed on the support. A covering layer 7 defines an aperture 4 that opens into a recessed space or well 8 having walls defined by layer 7 and a bottom defined by layer 6. As shown, the electrodes 2a, 2b, and 2c are situated in well 8. The electrodes are left exposed in well 8, such that sample fluid can be received in well 8 to contact the electrodes. The working electrode 2a comprises a coating of the enzyme-polymer composition crosslinked to a conductive electrode material, such as referenced herein. The counter electrode 2b is a conductive electrode material without the coating of the enzyme-polymer composition. The electrode support 6, typically an elongated strip of electrical insulating polymeric material, e.g., PVC, polycarbonate or polyester, supports two or more printed tracks of electrically conducting carbon ink 5. The conducting inks 5 are hidden in the view of FIG. 5, and are represented by hatched lines. These printed tracks define the positions of the working, counter, and reference electrodes, and of the electrical contacts 3 that are operable to be inserted into an appropriate measurement device (not shown). The covering layer 7 also can be an electrical insulating polymeric material. The insulating layers 6 and 7 can be, for example, hydrophobic insulating polymeric material. FIG. 6 further shows the electrodes as positioned in the well 8, where they can be contacted and covered by fluid sample during measurements. In addition to the arrangement shown, the working, counter, and reference electrodes can be arranged in other configurations relative to each other within recess well 8. The working, reference and counter electrodes can be spaced, for example, from about 0.25 mm to about 0.5 mm, and the working, counter, and reference electrodes can have a width, for example, of about 0.5 mm to 1.5 mm, and a length, for example, of from about 1.5 mm to about 2.5 mm, or other dimensions.

Methods and Systems for detecting, monitoring and treatment. The amperometric biosensor of the present invention can be incorporated in any system where a sample fluid can be contacted. The present bionsensor can be integrated, for example, into a flow-through system for creatinine determination. Such a system includes dialysis systems, such as hemodialysis and peritoneal dialysis systems. The present biosensor can provide information, for example, regarding the amount of creatinine in a fluid stream of a dialyzer used in the dialysis of an animal for measuring the creatinine concentration in the fluid stream in real time. The fluid stream of the dialyzer can be, for example, a dialysate stream, an arterial line, a venous line, or any combination thereof. The biosensor can be deployed, for example, in a dialysate stream (e.g., a post-dialyzer stream or a pre-cartridge stream). The biosensor can be used at a point of care, using a small quantity of the patient's blood or other bodily fluid (e.g., a glucose meter, a lactate meter).

Normal physiological creatinine concentration can be, for example, about 40 μM to 150 μM. Blood levels greater than, for example, 150 μM can indicate the need to perform tests such as “creatinine clearance.” By comparing the blood and urine levels of creatinine, the kidney function can be screened and the result is referred to as creatinine clearance. When the creatinine clearance falls to about 10-12 cc/minute, for example, the patient can be considered in need of dialysis. Creatinine concentrations are also commonly measured in units of milligrams per deciliter (mg/dl). Normal levels of creatinine in the blood are, for example, approximately 0.6 to 1.2 mg/dl in adult males and 0.5 to 1.1 mg/dl in adult females. Creatinine levels that reach or exceed, for example, 500 μM, or 10.0 mg/dl or more, in adults also can indicate severe kidney impairment and the need for dialysis machine to remove wastes from the blood.

A biosensor of the present invention can be used, for example, to determine a patient's creatinine level quickly and in real time as a screening measure, which can be used in lieu of or supplemental to conventional creatinine clearance tests. For example, the present biosensor can be used to directly determine creatinine concentration in a biological fluid obtained directly from the patient. The biosensor also can be used as part of a dialysis system or other treatment regimen used to clear waste, such as creatinine, from the patient's blood, for purposes of monitoring the progress of the treatment on the patient. For purposes of this present application, the terms “patient” or “subject” refers to any animal, e.g., a human or other mammalian animal. The monitoring and treatment location is not limited, and can encompass any site for monitoring and treatment, whether out-patient (e.g., at home), in-patient (e.g., at a hospital or other medical care facility), research lab or clinic, or other settings.

Dialysis is a treatment that removes the waste products and excess fluid that accumulate in the blood as a result of kidney failure. Chronic renal failure can occur when the renal function has deteriorated, for example, to about 25% of normal. This amount of deterioration can cause significant changes in the blood chemistry and is about the time that people feel poorly enough that they seek medical care. Dialysis is a treatment option for such conditions. Dialysis systems include, for example, hemodialysis and peritoneal dialysis systems. With peritoneal dialysis (PD), a mild saltwater solution containing dextrose and electrolytes called dialysate is put into the peritoneal cavity. Because there is a rich blood supply to this abdominal cavity, urea and other toxins from the blood and fluid are moved into the dialysate, thereby cleaning the blood. The dialysate is then drained from the peritoneum. Later “fresh” dialysate is again put into the peritoneum. Also, there is hemodialysis. This is a method of blood purification in which blood is continually removed from the body and passed through a dialyzer (artificial kidney) where metabolic waste and excess water are removed and pH and acid/base balance are normalized. The blood is simultaneously returned to the body. The dialyzer is a small disposable device consisting of a semi-permeable membrane. The membrane allows the wastes, electrolytes, and water to cross but restricts the passage of large molecular weight proteins and blood cells. Blood is pumped across one side of the membrane as dialysate is pumped in the opposite direction across the other side of the membrane. The dialysate is highly purified water with salts and electrolytes added. The machine is a control unit which acts to pump and control pressures, temperatures, and electrolyte concentrations of the blood and the dialysate. The average length of one hemodialysis treatment is 3-5 hours, or can be other durations. Several types of hemodialysis are generally known, which include single pass-hemodialysis and sorbent dialysis. Additional information and details on dialysis and these types of dialysis systems thereof are provided, for example, in U.S. Pat. No. 7,033,498, which are incorporated in their entirety herein by reference.

A dialysis system, for example, can be designed or adapted to have a dialysate stream evaluated with a biosensor as illustrated herein, such as to monitor creatinine clearance in the patient undergoing dialysis treatment. By determining the creatinine concentration of the patient receiving dialysis, as continuously, semi-continuously, or intermittently, in real time using the biosensor, the current progress and effectiveness of the treatment and state of the patient is accurately and rapidly known. By selecting a target value for creatinine clearance, for example, the system also optionally can be equipped with the ability to automatically record and inform the patient or health care provider of the progress towards and/or reaching of the desired clearance.

A calibration curve for electrical current and creatinine concentration can be generated for the biosensor, such as using standardized creatinine solutions of known concentrations with the biosensor and correlating detected current signals using the standards to generate a calibration curve for the biosensor. The calibration curve can be used for the analyses of fluid samples having unknown concentrations of creatinine.

Calibration curves for the sensors would be generated via amperometric testing where the operating potential is held constant and the resulting current at the working electrode is monitored as a function of time. At known times the test media will be spiked with known concentrations of the target analyte/s. The response, response time, and stability of response would all be characterized during these tests. Stable response currents would be graphed against the resulting analyte concentration to generate calibration curves.

The dialysis treatment can comprise, for example, contacting a present biosensor with a dialysate stream of a dialyzer as used in the dialysis of an animal for measuring the creatinine concentration in a dialysate stream in real time. The biosensor can be deployed in a dialysate stream post-dialyzer or pre-cartridge. The biosensor also can be used, for example, in the arterial line, venous line, or both, of the dialysis machine. The biosensor can be used to determine a patient's initial creatinine concentration, the progress of creatinine clearance during dialysis treatment, or both, in real time. The dialysis treatment of the animal can be discontinued after the measured creatinine concentration reaches a pre-selected target value. The creatinine concentration measurement can be done in the dialysate stream in real time continuously, semi-continuously, or intermittently. Further, where a correlation exists between creatinine and another chemical of interest, then it may be possible to determine these levels and clearance indirectly yet accurately with the biosensor. For example, the concentration of creatinine measured may be correlated to BUN (blood urea nitrogen), for evaluation of kidney function and/or monitoring the effectiveness of dialysis.

The biosensor of the present invention can be used with a variety of different commercially available dialysis machines, such as, but not limited to, peritoneal dialysis and hemodialysis systems (e.g., single-pass dialysis machines). One example is the ALLIENT dialysis system by Renal Solutions, Inc. The invention may also be used with any number of monitors, which can detect the difference between saline and blood, essentially anything measuring physical differences between saline and blood.

FIG. 7 is a schematic of an exemplary non-limiting dialysis system 10 with which a biosensor of the present invention can be used. Many features of the dialysis system of FIG. 7 are shown in U.S. Pat. Application Publication No. 2008/0149563 A1, which are incorporated in their entirety herein by reference. Referring to FIG. 7, the system 10 is a renal dialysis system for the extracorporeal treatment of blood from a patient 11 whose kidney function is impaired. The illustrated embodiment of the dialysis system 10 comprises a dialysis machine 12 as is generally known in the medical arts, and shown generally within the dotted line, plus various consumables as is known in the art. The dialysis system 10 is adapted to integrate a biosensor (1) of embodiments of the present invention, such as in a dialysate stream post-dialyzer or pre-cartridge stream (32′), or in other dialysis system fluid streams, as discussed in more detail hereinbelow. The sensor can be arranged, for example, in-line, such as arranged perpendicular to the direction of fluid flow.

The dialysis system 10 comprises a blood circuit 28 through which the patient's blood travels, a dialyzer 30 that serves to separate the wastes from the blood, and a dialysate circuit 32 through which treatment fluid, specifically dialysate, travels carrying the waste away. The dialysate is fed as a stream to the dialyzer 30 through feed line 32″ and the spent dialysate stream is discharged from dialyzer 30 into line 32′ of circuit 32. The feed line 32″ can be, for example, fresh or highly pure dialysate. The dialysis circuit 32 can further comprise, for example, a filter cartridge 35, such as loaded with an ultrafiltration (UF) filtration membrane and/or other filtering means, including any filtering means useful for regenerating a post-dialyzer dialysate stream back into fresh or highly pure dialysate that can be recirculated back to the dialyzer 30 throughout dialysis treatment. The filter cartridge 35 can be a commercial product, for example, such as a SORB™ or HISORB™ sorbent cartridge, available from Renal Solutions, Inc. The dialysate circuit 32 includes a dialysate pump 34 for driving dialysate fluid through the filter cartridge 35 and through the dialyzer 30. The pump also can be located, for example, at other locations in circuit 32. The dialysate circuit 32 may further include other components, system arrangements, and modes of operation, such as those described, for example, in U.S. Patent Application Publication No. 2005/0274658 A1 and U.S. Pat. No. 7,033,498, which are hereby incorporated by reference in their entireties.

The blood circuit 28 includes another tube set including an arterial line 36 for withdrawing blood from the patient 11 and delivering it to the dialyzer 30, and a venous line 38 for returning the treated blood to the patient 11. A blood pump 40 drives the blood around the blood circuit 28. A valve 41 is situated on a gas line 42 for supplying negative and positive pressure from a source 43 to the pump 40. The arterial line 36 also incorporates a valve 45 that can stop the flow of blood from the patient 11, an ultrasound or other monitor 46 of the type available from Transonic to measure the concentration of saline in the blood, and a flow sensor 47 that measures the flow of blood. The arterial line 36 further includes a valve 48 upstream of the pump 40 and a valve 50 downstream on the pump 40. The blood pump 40 may be configured as described in U.S. patent application Ser. No. 10/399,128, entitled Device and Methods for Body Fluid Flow Control In Extracorporeal Fluid Treatments, filed on Jul. 28, 2003, which is hereby incorporated by reference in its entirety.

Other components which interact with the blood circuit 28 include a source of fluid, such as a saline bag 52, which communicates with the arterial line 36 via a branch line 54 and a valve 56 responsive to processor 14. Additionally, an anticoagulant solution such as a heparin supply 58 may communicate with the arterial line 36 through a branch line 60 and a pump 62 responsive to processor 14. A saline bolus may be administered to the blood stream by briefly closing clamp 45 opening clamp 56 and continuing operation of blood pump 40, thus drawing in saline rather than blood into the circuit. The clamps may then be returned to position for the pump to draw blood into the circuit and push the saline and blood through the dialyzer and return blood line 38. It is understood by persons skilled in the art that additional elements may be added to the blood circuit 36, such as air detectors in the branch lines 54 or 60. These additional elements are omitted from the drawings for clarity of illustration. Finally, the venous line 38, which delivers the treated blood from the dialyzer 30 to the patient 11, also includes a valve 64, an ultrasound monitor 66 of the type available from Transonic, and a flow sensor 68.

The dialysis machine 12 may be provided with a non-volatile memory component 16 adaptively coupled to an electronic control means 14, which may be a processor. Non-volatile memory component 16 can be any form of memory component that retains stored values when external power is turned off. The memory 16 may store instruction which, when executed, perform the various embodiments of the disclosed method.

Dialysis machine 12 further includes a data entry device 18, such as a keyboard, touch-screen monitor, computer mouse, or the like. Dialysis machine 12 further includes a display device 20, such as a read-out monitor, for displays of operating values of the various individual components of the dialysis machine 12. The system 10 can be provided with a power source 22, a battery back-up 24, and a clock/timer 26. The processor 14, memory 16, data entry device 18, and clock/timer 26 represent one configuration of a control system.

The processor 14 coordinates the operation of the dialysis system 10 by controlling the blood flow in the blood circuit 28, the dialysate flow in the dialysate circuit 32, and the flow of saline 52 or heparin 58 to the arterial line 36 via the branch lines 54 and 60, respectively. To achieve this, the processor 14 utilizes hardware and/or software configured for operation of these components and may comprise any suitable programmable logic controller or other control device, or combination of control devices, that is programmed or otherwise configured to perform as is known in the art. Thus, blood flow in the blood circuit 28 is controlled by operating the blood pump 40 and controlling the valves in the arterial and 36 and venous 38 lines. Dialysate flow in the dialysate circuit 32 is controlled by operating the dialysate pump 34. The processor 14 is also responsive to various input signals it receives, such as input signals from one or more flow sensors 47, 68, ultrasound monitors 46, 66, and the clock/timer 26. Note that ultrasonic transit time monitors can serve both for measurement of flow and measurement of saline concentration within the blood. Thus, the function of sensors 46 and 47 may be provided by a single sensor and the function of sensors 64 and 68 may also be provided by a single sensor. Additionally, the processor 14 displays system status and various other treatment parameters, known in the art, on the display 20. That allows the operator to interact with the processor 14 via the data entry device 18 (which could include a touch sensitive display 20).

The sensing portion of the biosensor 1 is arranged to be contacted with dialysate fluid in a dialysate stream, which can be, for example, a post-dialyzer dialysate stream or a pre-cartridge dialysate stream (32′). Feature 100 generally refers to a signal processing system, which comprises signal processing components and arrangements (not shown), which can be used with the biosensor. Signal processing arrangements for biosensors are conventionally known that can be adapted for use with a biosensor of the present invention. The signal processing systems can include, for example, a working voltage generating circuit arranged to apply a working voltage to the working electrode, a controller which causes the working voltage generating circuit to apply a voltage to the working electrode, an A/D converter for converting the analog current signal into a digital current signal, an amplifier, and/or other components conventionally used or useful in signal processing systems for biosensors. The acquired signals from the biosensor can be transmitted to the processor 14 for analysis. A calibration curve stored in memory (16) can be used, for example, in conjunction with the processor 14 to make a real-time calculation of the creatinine concentration of the sensed sample of the dialysate stream based on the current signal acquired by the biosensor 1.

As an optional additional or alternative creatinine biosensing location used in the dialysis system shown in FIG. 7, a fluid stream of the dialyzer that is analyzed for creatinine concentration also can be in the blood circuit 28, such as, for example, in sensor location 101′ in the arterial line, in sensor location 101 in the venous line, or both. Features 1001′ and 1001 are signal processing systems associated with sensor locations 101 and 101′ that can be similar to system 100, which is discussed above.

As stated above, the creatinine concentration of the patient receiving dialysis can be determined continuously, semi-continuously, or intermittently, in real time using the present biosensor in such an exemplified dialysis system. As stated above, the dialysis system can be configured such that when a pre-selected creatinine clearance level is achieved on a patient during a dialysis treatment, the patient and/or health care professional can be alerted by the system, such via text and/or graphics displayed on the display, and/or with an audio prompter, to notify of the progress of the treatment and status of the patient.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

It will be apparent to those skilled in the art that various modifications and variations can be made to the embodiments of the present invention without departing from the spirit or scope of the present invention. Thus, it is intended that the present invention covers other modifications and variations of this invention provided they come within the scope of the appended claims and their equivalents.

What is claimed is:

1. An amperometric biosensor for determination of creatinine in a sample fluid comprising an enzyme-polymer composition and an electrode having a surface, wherein the enzyme-polymer composition comprises at least one redox polymer and a plurality of enzymes immobilized on the electrode surface, and wherein the enzymes comprise at least one redox enzyme and at least one enzyme catalyzing hydrolysis of creatinine or a hydrolyzed derivative thereof.

2. The biosensor of claim 1, wherein the enzyme-polymer composition is a coating on the surface of the electrode.

3. The biosensor of claim 1, where the redox polymer is attached to the enzymes and the electrode surface of the biosensor through crosslinking.

4. The biosensor of claim 1, wherein the redox polymer comprises a neutral polymeric backbone and redox active moieties attached thereto.

5. The biosensor of claim 1, wherein the redox polymer comprises a neutral polymeric backbone and redox active moieties attached thereto, wherein the redox moieties comprise organometallic species comprising a transition metal.

6. The biosensor of claim 1, wherein said redox polymer is X-poly(vinylpyridine), X-poly(vinylimidazole), X-poly(allylamine), or X-poly(ethyleninime) or any combination thereof, where X is at least one organometallic moiety comprising a transition metal that is iron, osmium, ruthenium, or cobalt or any combination thereof.

7. The biosensor of claim 1, wherein said plurality of immobilized enzymes comprise creatinine amidohydrolase, creatine amidinohydrolase, and sarcosine oxidase.

8. The biosensor of claim 1, wherein said composition contains from about 1 wt % to about 99 wt % redox polymer, from about 1 wt % to about 99 wt % enzymes, and from about 1 wt % to about 30 wt % crosslinker, wherein the enzymes comprise creatinine amidohydrolase, creatine amidinohydrolase, and sarcosine oxidase.

9. The biosensor of claim 1, wherein said biosensor comprises at least one working electrode, at least one reference electrode and at least one counter electrode.

10. The biosensor of claim 1, wherein said biosensor comprises at least one working electrode, at least one reference electrode and at least one counter electrode, and wherein the enzyme-polymer composition is applied to said working electrode.

11. A dialysis system comprising the amperometric biosensor of claim 1.

12. An immobilized enzyme-polymer composition for an electrode surface comprising at least one crosslinked redox polymer and a plurality of enzymes comprising at least one redox enzyme and at least one enzyme catalyzing hydrolysis of creatinine or a hydrolyzed derivative thereof.

13. A method for making the amperometric biosensor of claim 1 comprising:
depositing an aqueous mixture containing said plurality of enzymes, at least one redox polymer and at least one crosslinker on a surface of said electrode; and
crosslinking the mixture to form said enzyme-polymer composition immobilized on the electrode surface.

14. The method of claim 13, wherein said plurality of enzymes comprise creatinine amidohydrolase, creatine amidinohydrolase, and sarcosine oxidase.

15. The method of claim 13, wherein said redox polymer is X-poly(vinylpyridine), X-poly(vinylimidazole), X-poly(allylamine), or X-poly(ethyleninime) or any combination thereof, where X is at least one organometallic moiety comprising a transition metal that is iron, osmium, ruthenium, or cobalt, or any combination thereof.

16. A method of detecting creatinine concentration in a sample fluid, comprising:
contacting the biosensor of claim 1 with a sample fluid;
measuring current at the electrode; and
correlating the measured current with creatinine concentration in the sample fluid.

17. The method of claim 16, wherein the sample fluid is a dialysate stream.

18. The method of claim 16, wherein the sample fluid is a biological fluid.

19. The method of claim 16, wherein the electrode is operated at from about 350 to about 400 mV.

20. A method of treating an animal for clearance of creatinine, comprising:
contacting the biosensor of claim 1 with a fluid stream of a dialyzer used in the dialysis of an animal; and
measuring creatinine concentration in said fluid stream of the dialyzer with the biosensor.

21. The method of claim 20, wherein the fluid stream used for the creatinine concentration measurement is a dialysate stream.

22. The method of claim 20, wherein the fluid stream used for the creatinine concentration measurement is a post-dialyzer dialysate stream.

23. The method of claim 20, where the creatinine concentration measurement is done in real time continuously, semi-continuously, or intermittently.

24. The method of claim 20, further comprising discontinuing dialysis treatment on the animal after a measured creatinine concentration reaches a pre-selected target value.
